(19) United States
(12) Patent Application Publication
(10) Pub. No.: US 2019/0117807 A1
(43) Pub. Date: Apr. 25, 2019

(54) DEVICE FOR STERILIZING AN OBJECT, THE DEVICE HAVING A STREAM DEFLECTOR

(71) Applicant: SOCIETE POUR LA CONCEPTION DES APPLICATIONS DES TECHNIQUES ELECTRONIQUES, Merignac (FR)

(72) Inventors: Thierry CASABONNE, Bordeaux (FR); Sandrine VILLEGER, Montjoire (FR); Marie-Agnès BENOIT, Merignac (FR)

(21) Appl. No.: 15/848,379

(22) Filed: Dec. 20, 2017

(30) Foreign Application Priority Data
Oct. 23, 2017 (FR) 1759979

(51) Int. Cl. A61L 2/14; A61L 2/24; H05H 1/24

(52) U.S. Cl. CPC A61L 2/14; H05H 2245/1225; H05H 1/24; A61L 2/24

(57) ABSTRACT

A sterilization device for sterilizing an object comprises a sterilization chamber defining a treatment zone in which the object for sterilizing is to be positioned; a nitrogen source; a plasma generator in communication with the nitrogen source; and a duct putting the plasma generator into communication with the sterilization chamber and through which a post-discharge stream resulting from a plasma produced by the plasma generator is to flow towards the sterilization chamber. The duct defines an injection orifice for injecting the post-discharge stream into the sterilization chamber. The device has a deflector for deflecting the post-discharge stream and positioned in the sterilization chamber facing a central portion of the injection orifice, upstream from the treatment zone. The deflector is configured to deflect the flow of a central portion of the post-discharge stream as introduced through the central portion of the injection orifice.

The present invention relates to a device for sterilizing an object with a post-discharge stream resulting from a plasma produced by a plasma generator, and the invention also relates to a method of sterilization using such a device.

### BACKGROUND OF THE INVENTION

It is known to sterilize objects by means of an autoclave in which the object to be sterilized is raised to a determined high temperature, of the order of 120° C., and for this to be done during determined time periods with cycles that are specified by legislation.

Applying a temperature higher than 70° C. can raise problems and lead to certain objects being damaged, e.g. when the object includes portions made of polymer materials.

Methods enabling sterilization to be performed at lower temperatures have consequently been developed in order to reduce the damage to objects while they are being treated.

In this context, devices have been proposed that make use of a post-discharge stream from a plasma. The post-discharge from a plasma comprises neutral species resulting from the ions of the plasma that is formed recombining with electrons. Those devices enable the object to be sterilized with the post-discharge stream resulting from the plasma produced by a plasma generator. Nevertheless, the object can be damaged during treatment, even with that type of device. When the object is present in packaging during the treatment, it has been found that the packaging can also be damaged in that type of device.

Still for the purpose of reducing damage to the object or to its packaging, proposals have been made in the prior art to avoid using oxygen when generating the plasma, and to make the plasma by using nitrogen. The use of nitrogen as gas for forming the plasma is described in particular in Document U.S. Pat. No. 7,927,557. The technique implemented in that document leads to good results in terms of sterilization, and does indeed enable damage to the object and to its packaging to be reduced during the process of sterilization by the post-discharge stream resulting from the plasma that is generated.

Nevertheless, it remains desirable to further improve methods of sterilizing objects with a post-discharge stream resulting from a nitrogen plasma in order to obtain satisfactory sterilization without damaging the treated object or the packaging in which the object is present during sterilization.

### OBJECT AND SUMMARY OF THE INVENTION

In a first aspect, the invention provides a sterilization device for sterilizing an object, the device comprising at least:

a sterilization chamber defining a treatment zone in which the object for sterilizing is to be positioned;

a nitrogen source;

a plasma generator in communication with the nitrogen source; and

a duct putting the plasma generator into communication with the sterilization chamber and through which a post-discharge stream resulting from a plasma produced by the plasma generator is to flow towards the sterilization chamber, the duct defining an injection orifice for injecting the post-discharge stream into the sterilization chamber;

the device being characterized in that it further comprises a deflector for deflecting the post -discharge stream and positioned in the sterilization chamber facing a central portion of the injection orifice, upstream from the treatment zone, the deflector being configured to deflect the flow of a central portion of the post-discharge stream as introduced through the central portion of the injection orifice.

The terms “upstream” and “downstream” are used herein relative to the flow direction of the post-discharge stream towards the treatment zone.

The inventors have found that the central portion of the post-discharge stream is significantly more aggressive than the peripheral portion of the post-discharge stream surrounding the central portion.

Specifically, firstly the central portion has a greater concentration of metastable species than does the peripheral portion, which metastable species constitute species that are aggressive relative to the object for treatment or relative to the packaging in which the object is present. This difference in concentration can be explained by the fact that the metastable species in the peripheral portion have a greater probability of disappearing as a result of collisions with the wall of the duct, given that they are closer to that wall. Secondly, the species in the central portion follow a straight line path at a speed that is greater than the speed of the species that are present in the peripheral portion, thereby giving them higher kinetic energy that might damage the object for treatment or its packaging on impacting thereagainst.

The invention proposes positioning a deflector constituting an obstacle for the flow in the central portion of the post-discharge stream, upstream from the treatment zone. The flow in the central portion of the post-discharge stream is thus deflected by the deflector before penetrating into the treatment zone where the object is positioned. During such deflection, the aggressive species present in the central portion are destroyed by colliding with the deflector. This deflection also serves to reduce the speed of the species present in the central portion and to “break” their straight line paths. These effects serve to reduce significantly the damage to the object for sterilizing during its treatment or the damage to the packaging in which the object is present. Furthermore, the presence of the deflector contributes to reducing the temperature of the post-discharge stream reaching the object, thereby also contributing to reducing the risks of damage.

In a first example, the deflector constitutes an obstacle positioned facing the central portion of the injection orifice so as to cause the post-discharge stream to flow around it.

In particular, the injection orifice may have first and second lateral portions situated on either side of the central portion of the injection orifice, the deflector not being positioned facing the first and second lateral portions.

Such a characteristic serves advantageously to obtain more uniform introduction of the post -discharge stream into the treatment zone.

In a second example, the deflector comprises at least one multiply-perforated wall in communication with the injection orifice and leading into the treatment zone, the multiply -perforated wall being designed to have the post-discharge stream pass therethrough and defining, upstream from the treatment zone, an internal volume for distributing the post-discharge stream, the deflector having a non-perforated portion positioned facing the central portion of the injection orifice.

The non-perforated portion serves to deflect the flow in the central portion of the post -discharge stream so as to reduce the aggressive nature of the post-discharge stream relative to the object for treatment or relative to its packaging. The use of a multiply-perforated wall makes it possible to perform multi-point injection of the post-discharge stream into the treatment zone, thus making it possible to obtain more uniform introduction of the post-discharge stream into this zone.

In particular, the deflector may further comprise a guide portion for guiding the post -discharge stream and defining an internal channel putting the injection orifice into communication with the multiply-perforated wall.

In an example, the internal channel presents a varying section that increases on going towards the multiply-perforated wall.

This increase in the section of the internal channel enables it to define a zone where pressure is lower than in the duct. The arrival of species in this reduced pressure zone slows them down, thereby still further reducing their aggressive nature, and increases their mean free paths, further improving their diffusion in the treatment zone.

In an example, the multiply-perforated wall has a first end situated beside the injection orifice and a second end opposite from the first end, the size of the perforations in the multiply -perforated wall increasing on going from the first end towards the second end.

Under such circumstances, the perforations furthest away from the injection orifice (situated beside the second end) are larger than the orifices closer to the injection orifice (situated beside the first end). This makes it possible to obtain even more uniform introduction of the post -discharge stream into the treatment zone.

In particular, the treatment zone may have a support surface on which the object for sterilizing is to be positioned, the multiply-perforated wall overlying the support surface and opening out facing it.

In certain circumstances, the objects for sterilizing are packaged in packages that are permeable to the sterilizing species but that do not allow pathogens to penetrate, and that present greater porosity over the top of the object for sterilizing. The fact that the multiply-perforated wall overlies the support surface and opens out facing it enables the post-discharge stream to be injected through the high porosity surface of the packaging, thereby improving the effectiveness of sterilization when sterilizing packaged objects.

In an embodiment, the object is a medical instrument.

The present invention also provides a method of sterilizing an object by implementing a device as described above, the method comprising a step of sterilizing the object present in the treatment zone, during which the object is treated by the post-discharge stream injected through the injection orifice.

### BRIEF DESCRIPTION OF THE DRAWINGS

Other characteristics and advantages of the invention appear from the following description given in non-limiting manner with reference to the accompanying drawings, in which:

FIG. 1 is a diagram showing an example of a sterilization device of the invention;

FIG. 2 is a diagram showing the flow of the post-discharge stream at the deflector in the FIG. 1 device;

FIG. 3 is a diagrammatic section view at the injection orifice in the FIG. 1 device;

FIGS. 4 to 6 are diagrams showing deflector variants suitable for use in the context of the invention; and

FIG. 5A is a diagrammatic view showing a variant multiply-perforated wall suitable for use in the example of FIG. 5.

### DETAILED DESCRIPTION OF EMBODIMENTS

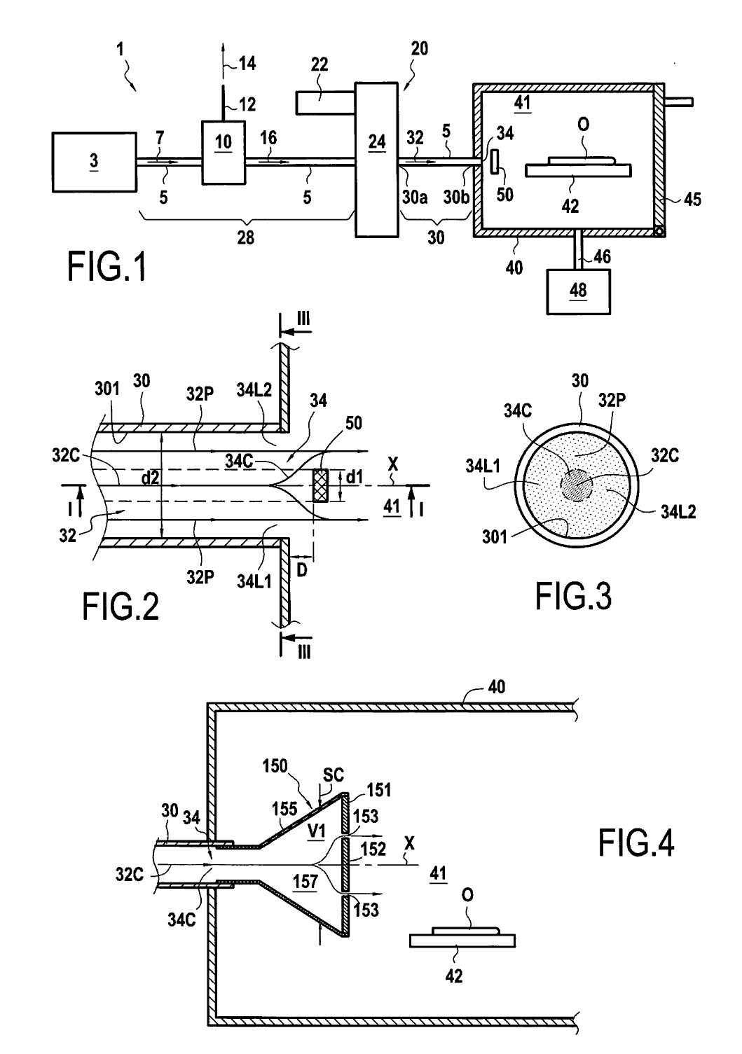

FIG. 1 is a diagram showing a sterilization device 1 configured to sterilize an object O by treatment with a post-discharge stream resulting from a nitrogen plasma.

The device 1 comprises a duct 5 having a first segment 28 putting a compressor 3 into communication with a plasma generator 20. The first segment 28 has a nitrogen filter element 10 situated between the compressor 3 and the plasma generator 20.

A stream of compressed air 7 coming from the compressor 3 flows through the first segment 28 to the filter element 10. The filter element 10 is constituted by a known element that is configured to separate nitrogen from oxygen in the compressed air stream 7. After passing through the filter element 10, a nitrogen stream 16 flows through the first segment 28 to the plasma generator 20. The oxygen 14 that is separated from the nitrogen is discharged via an exhaust duct 12.

The first segment 28 serves to deliver the nitrogen stream 16 to the plasma generator 20. In known manner, the plasma generator 20 serves to generate a nitrogen plasma from the nitrogen stream 16. The plasma generator 20 comprises an evacuated enclosure 24 subjected to the action of an electromagnetic field, constituted in this example by a microwave generator 22. The electromagnetic field generated in the enclosure 24 presents intensity that is high enough to cause nitrogen to ionize.

The duct has a second segment 30 that puts the plasma generator 20 into communication with a sterilization chamber 40 in which the object O for sterilizing is positioned. The post -discharge stream 32 from the nitrogen plasma flows to the sterilization chamber 40 via the second segment 30.

The sterilization chamber 40 defines a treatment zone 41 including at least one support 42 on which the object O is positioned during the sterilization treatment. A treatment zone 41 is shown that has only one support 42 and only one object O, however it would naturally not go beyond the ambit of the invention for the treatment zone to have a plurality of supports each carrying one or more objects. The sterilization chamber 40 has a door 45 to enable the object O to be inserted into the treatment zone 41, and to be removed after sterilization.

The object O may be a medical instrument such as an endoscope, a chisel, or a scalpel. The invention is also advantageous for sterilizing objects other than medical instruments, such as electronic cards.

The second segment 30 presents a proximal end 30a situated beside the plasma generator 20 and in communication therewith. The second segment 30 also presents a distal end 30b defining an injection orifice 34 for injecting the post-discharge stream 32 and leading into the sterilization chamber 40. The plasma generated by the plasma generator 20 penetrates into the second segment 30 via the proximal end 30a. During the flow of the plasma that has been formed through the second segment 30, the ionic species of the plasma recombine with electrons so as to obtain the post-discharge stream 32. As mentioned above, the central portion of the post -discharge stream 32 is significantly more aggressive than the peripheral portion. The post -discharge stream 32 flows through the second segment 30 and is injected into the sterilization chamber 40 through the injection orifice 34. The treatment zone 41 is in communication with a vacuum pump 48. This pump drives the post-discharge stream 32 into the treatment zone 41 and ensures that gas is discharged to the outside via a second duct 46.

FIGS. 2 and 3 show the central and peripheral portions of the post-discharge stream 32 injected through the injection orifice 34. The post-discharge stream 32 is injected into the sterilization chamber 40 through the injection orifice 34 along an injection direction X. During sterilization, the object O may be subjected to a post-discharge stream that is continuous, or in a variant, to a post-discharge stream that is pulsed.

The peripheral portion 32P of the post-discharge stream 32 surrounds the central portion 32C of the post-discharge stream 32. The peripheral portion 32P is less charged with metastable species than is the central portion 32C.

The injection orifice 34 presents a central portion 34C and first and second lateral portions 34L1 and 34L2 situated on either side of the central portion 34C.

The central portion 34C of the injection orifice 34 is situated between the first and second lateral portions 34L1 and 34L2 of the injection orifice 34. The central portion 34C of the injection orifice 34 constitutes the portion through which the central portion 32C of the post -discharge stream 32 passes. The lateral portions 34L1 and 34L2 have the peripheral portion 34P of the post-discharge stream 32 passing therethrough.

FIGS. 1 and 2 show a first example of a deflector 50 usable in the context of the invention. As mentioned above, the deflector 50 is positioned facing the central portion 34C of the injection orifice 34 and upstream from the treatment zone 41 so as to deflect the flow of the central portion 32C of the post-discharge stream 32 before it is introduced into the treatment zone 41.

The deflector 50 shown in FIGS. 1 and 2 is constituted by a solid (non-perforated) wall positioned facing the central portion 34C of the injection orifice 34 and serving to cause the post -discharge stream 32 to flow around it. The deflector 50 constitutes an obstacle for the central portion 32C of the post-discharge stream 32 and serves to deflect it before it reaches the object O for treatment. In variants that are described below with reference to FIGS. 4 to 6, the deflector is not designed to cause the post-discharge stream to flow around it but rather to have the stream flow through it.

In this example, the deflector 50 is fastened to a wall of the sterilization chamber 40, e.g. by screw-fastening. The deflector 50 may be made of ceramic material, e.g. of alumina. Advantageously, the deflector 50 is positioned in the vicinity of the injection orifice 34 or indeed at zero distance therefrom. By way of example, the deflector 50 is positioned at a distance D from the injection orifice 34 as measured along the injection direction X that is less than or equal to 50 millimeters (mm). The deflector 50 constitutes an element that is distinct from the support 42 on which the object O is present. The deflector 50 may be elongate in shape, extending transversely relative to the injection direction X of the post-discharge stream 32. By way of example, the deflector may be in the shape of a rectangular parallelepiped or of a cylinder. Nevertheless, it would not go beyond the ambit of the invention for the deflector to be of some other shape, such as for example in the shape of a disk or of a sphere.

As mentioned above, the deflector 50 in this example is intended to make the post-discharge stream 32 flow around it. In this example, the deflector 50 presents at least one dimension d1, e.g. its greatest transverse dimension, that is less than the greatest dimension d2 of the injection orifice 34. Thus, the deflector 50 shown in this example does not constitute an obstacle for at least a fraction of the peripheral portion 32P of the post-discharge stream 32.

In particular, the deflector 50 may be centered on the central portion 32C of the post -discharge stream 32 entering via the injection orifice 34. In the example shown, the deflector 50 faces neither the first lateral portion 34L1 of the injection orifice 34 nor the second lateral portion 34L2 of the injection orifice 34. Thus, the peripheral portion 32P of the post-discharge stream 32 flowing through the lateral portions 34L1 and 34L2 is not deflected by the deflector 50. Nevertheless, it would not go beyond the ambit of the invention if the arrangement were different. In particular, in a variant that is not shown, the deflector could face the central portion 34C of the injection orifice 34 and at least one of its lateral portions 34L1 or 34L2.

The inventors have tested sterilization of the object O while using a device 1 as shown in FIG. 1. In that test, the nitrogen plasma was generated by a “Surfaguide” plasma generator 20 using a magnetron having a power of 300 watts (W) emitting a magnetic field at a frequency of 2450 megahertz (MHz). In that test, the length of the second segment 30 was 206 mm and an alumina deflector 50 of cylindrical shape having a diameter of 8 mm was positioned facing the central portion of the injection orifice 34 at a distance of 2 mm from the orifice 34. The test enabled satisfactory sterilization to be obtained without damaging the object.

The example device 1 shown in FIG. 1 has a single injection orifice 34. Naturally, it would not go beyond the ambit of the invention for the post-discharge stream to be injected through a plurality of injection orifices 34, with a respective deflector 50 being positioned facing each of the injection orifices 34.

The description above relates to a first example of a deflector 50 that causes the injected post-discharge stream 32 to flow around it. With reference to FIGS. 4 to 6, there follows a description of variant deflectors that are designed to pass the flow of the post-discharge stream that is introduced via the injection orifice.

The deflector 150 shown in FIG. 4 includes a wall 151 presenting a plurality of perforations 153. The perforations 153 are in communication with the injection orifice 34 and they lead into the treatment zone 41. The multiply-perforated wall 151 defines an internal volume V1 for distributing the post-discharge stream, which volume is situated upstream from the wall 151 and downstream from the injection orifice 34. The multiply-perforated wall 151 is situated between the treatment zone 41 and the internal volume V1. The multiply-perforated wall 151 is designed to have the post-discharge stream pass therethrough, which stream is to be distributed in the treatment zone 41 through the perforations 153.

In the presently-illustrated example, the multiply-perforated wall 151 extends transversely, e.g. perpendicularly, relative to the injection direction X. The wall 151 presents a non-perforated portion 152 situated facing the central portion 34C of the injection orifice 34. The non -perforated portion 152 is for deflecting the flow of the central portion 32C of the post-discharge stream 32 introduced through the injection orifice 34. As mentioned above, this serves to reduce the aggressive nature of the post-discharge stream relative to the object O for treatment or relative to its packaging. The portion of the post-discharge stream that is deflected in this way is subsequently distributed in the treatment zone 41 via the perforations 153.

As mentioned above, the multiply-perforated wall 151 serves to perform multi-point injection of the post-discharge stream into the treatment zone 41, thereby obtaining more uniform introduction of the post-discharge stream into this zone.

In the presently-illustrated example, the deflector 150 has a guide portion 155 that serves to guide the post-discharge stream as introduced through the injection orifice 34 towards the multiply-perforated wall 151.

The guide portion 155 defines an internal channel 157 situated extending the injection orifice 34 and putting the injection orifice 34 into communication with the multiply-perforated wall 151. The guide portion 155 may extend along the injection direction X. In this example, the guide portion 155 is flared in shape. The internal channel 157 presents a section SC that increases on going towards the treatment zone 41. In the presently-illustrated example, the guide portion 155 defines the internal volume V1 for distribution the post-discharge stream. The guide portion 155 is fastened to the injection orifice 34, e.g. by screw-fastening.

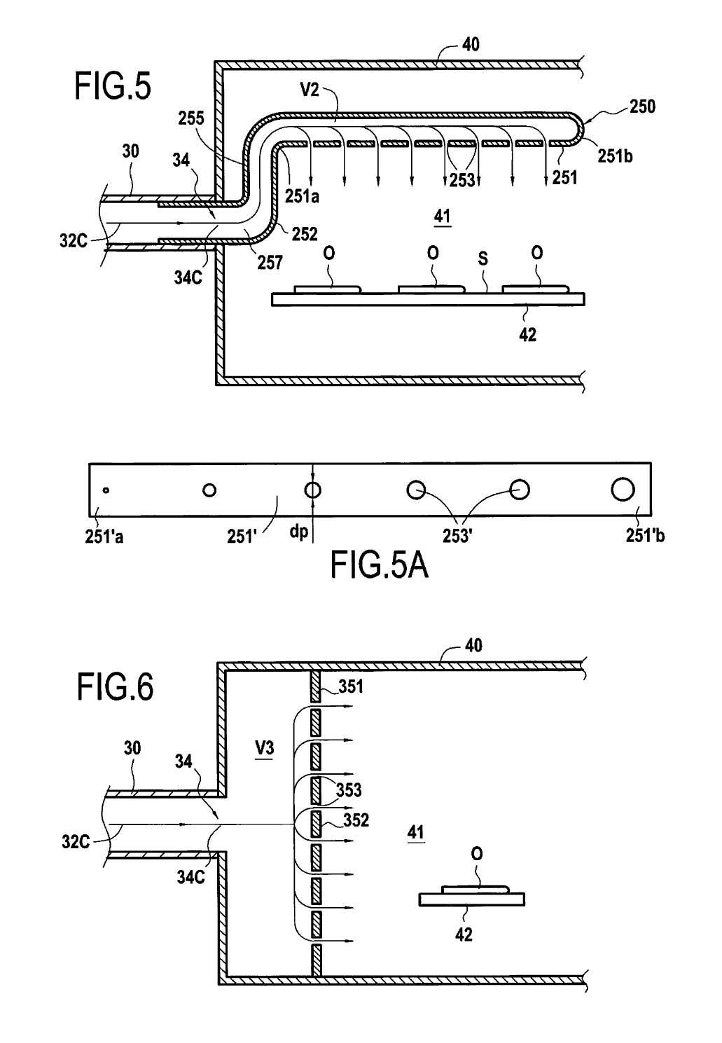

FIG. 5 shows another variant deflector 250 suitable for use in the context of the present invention and including a wall 251 having a plurality of perforations 253.

The wall 251 defines an internal volume V2 for distributing the post-discharge stream and situated upstream from the treatment zone 41. The multiply-perforated wall 251 is situated between the internal volume V2 and the treatment zone 41.

As in FIG. 4, the deflector 250 has a guide portion 255 putting the injection orifice 34 into communication with the perforations 253.

The guide portion 255 defines an internal channel 257 putting the injection orifice 34 into communication with the perforations 253. In the variant of FIG. 5, the guide portion 255 has a non-perforated portion 252 situated facing the central portion 34C of the injection orifice 34 and serving to deflect the central portion 32C of the post-discharge stream so as to reduce its aggressive nature relative to the object O for treatment or relative to the packaging in which the object is present.

In this variant, the multiply-perforated wall 251 extends transversely relative to the non -perforated portion 252. The multiply-perforated wall 251 has a first end 251a situated beside the injection orifice 34 and a second end 251b opposite from the first end 251a. In the example of FIG. 5, each of the perforations 253 has the same diameter.

FIG. 5A shows a variant in which the multiply-perforated plate 251′ has perforations 253′ of varying diameter. FIG. 5A shows how the diameter dₚ of the perforations 253′ varies along the multiply-perforated wall 251′. In the example shown, the diameter dₚ of the perforations 253′ increases on going away from the first end 251′a towards the second end 251′b.

In FIGS. 5 and 5A, the multiply-perforated wall 251 and 251′ overlies the support 42 on which the object(s) O is/are present and it opens out towards the support. In this variant, the post-discharge stream is injected into the treatment zone 41 transversely, e.g. perpendicularly, relative to the support surface S on which the object O is positioned. When the objects O are packaged, this injection thus takes place transversely to the surface of the packaging that presents high porosity, as situated above the object O, thereby enabling improved sterilization effectiveness to be obtained.

FIG. 6 shows another variant using a deflector having a multiply-perforated wall 351 through which the post-discharge stream is to pass, but without a guide portion.

The multiply-perforated wall 351 has a non-perforated portion 352 situated facing the central portion 34C of the injection orifice, thereby serving to deflect the central portion 32C of the post-discharge stream so as to reduce the aggressive nature of the post-discharge stream relative to the object O for treatment or relative to its packaging.

The wall 351 defines an internal volume V3 for distributing the post-discharge stream and in communication with the perforations 353 in the wall 351. In this example, the internal volume V3 for distributing the post-discharge stream is defined by the multiply-perforated wall 351 and by the sterilization chamber 40.

1. A sterilization device for sterilizing an object, the device comprising at least:
a sterilization chamber defining a treatment zone in which the object for sterilizing is to be positioned;
a nitrogen source;
a plasma generator in communication with the nitrogen source; and
a duct putting the plasma generator into communication with the sterilization chamber and through which a post-discharge stream resulting from a plasma produced by the plasma generator is to flow towards the sterilization chamber, the duct defining an injection orifice for injecting the post-discharge stream into the sterilization chamber;
wherein the device further comprises a deflector for deflecting the post-discharge stream and positioned in the sterilization chamber facing a central portion of the injection orifice, upstream from the treatment zone, the deflector being configured to deflect the flow of a central portion of the post-discharge stream as introduced through the central portion of the injection orifice.

2. A device according to claim 1, wherein the deflector constitutes an obstacle positioned facing the central portion of the injection orifice so as to cause the post-discharge stream to flow around it.

3. A device according to claim 2, wherein the injection orifice has first and second lateral portions situated on either side of the central portion of the injection orifice, the deflector not being positioned facing the first and second lateral portions.

4. A device according to claim 1, wherein the deflector comprises at least one multiply -perforated wall in communication with the injection orifice and leading into the treatment zone, the multiply-perforated wall being designed to have the post-discharge stream pass therethrough and defining, upstream from the treatment zone, an internal volume for distributing the post -discharge stream, the deflector having a non-perforated portion positioned facing the central portion of the injection orifice.

5. A device according to claim 4, wherein the deflector further comprises a guide portion for guiding the post-discharge stream and defining an internal channel putting the injection orifice into communication with the multiply-perforated wall.

6. A device according to claim 5, wherein the internal channel presents a varying section that increases on going towards the multiply-perforated wall.

7. A device according to claim 5, wherein the multiply-perforated wall has a first end situated beside the injection orifice and a second end opposite from the first end, the size of the perforations in the multiply-perforated wall increasing on going from the first end towards the second end.

8. A device according to claim 4, wherein the treatment zone has a support surface on which the object for sterilizing is to be positioned, the multiply-perforated wall overlying the support surface and opening out facing it.

9. A device according to claim 1, wherein the object is a medical instrument.

10. A method of sterilizing an object by using a device according to claim 1, the method comprising a step of sterilizing the object present in the treatment zone during which the object is treated by the post-discharge stream as introduced through the injection orifice.
